(19) United States
(12) Patent Application Publication
(10) Pub. No.: US 2011/0104123 A1
(43) Pub. Date: May 5, 2011

(54) THERAPEUTIC PROTOCOL USING STEM CELLS IN TISSUE AND NEURONAL REPAIR, MAINTENANCE, REGENERATION AND AUGMENTATION

(75) Inventor: Donald Newgreen, Victoria (AU)

(73) Assignee: Murdoch Childrens Research Institute Royal Children's Hospital, Victoria (AU)

(21) Appl. No.: 12/672,499

(22) PCT Filed: Aug. 8, 2008
(86) PCT No.: PCT/AU08/01159

§ 371 Date: Jan. 19, 2011

(30) Foreign Application Priority Data
Aug. 9, 2007 (AU) 2007904282

(51) Int. Cl. A61K 35/12; A61P 25/00; A61P 9/00

(52) U.S. Cl. 424/93.7

(57) ABSTRACT

The present invention relates generally to the field of tissue and neuronal repair, maintenance, regeneration and augmentation. More particularly, the present invention encompasses an improved stem cell therapeutic protocol.

### FIELD

The present invention relates generally to the field of tissue and neuronal repair, maintenance, regeneration and augmentation. More particularly, the present invention encompasses an improved stem cell therapeutic protocol.

### BACKGROUND

Bibliographic details of the publications referred to by author in this specification are collected at the end of the description.

Reference to any prior art in this specification is not, and should not be taken as, an acknowledgment or any form of suggestion that this prior art forms part of the common general knowledge in any country.

Stem cells provide enormous potential for use in therapeutic protocols to replace, repair or augment diseased, dysfunctional or absent cells (Kieburtz and Olanow, Mt Sinai J Med 74(1):7-14, 2007.

Stem cells are undifferentiated cells with long term viability, potential to replace themselves, but able to give rise to several lines of terminally differentiated cells. Embryonic stem (ES) cells such as mouse and human ES cells are derived from the inner cell mass at the earliest stage of embryogenesis (Biswas and Hutchins, Stem Cells Div 16(2):213-222, 2007. They are essentially totipotent, that is, their differentiation potential is unrestricted: they can form any type of cell under appropriate conditions. However, stem cells of potential clinical importance can be isolated from older embryonic, foetal and post-natal tissues (“older stem cells”) [Larru, Trends Biotechnol 19(12):487, 2001]. These cells occur in so-called stem cell niches in very low proportions relative to differentiated cells. In these niches they are mitotically inactive despite their enormous proliferative potential. In contrast to ES cells most older stem cells are restricted, in that although they will readily differentiate in some directions their competence in other directions is restricted.

This progressive restriction of potency indicates that these cells were allocated to the stem cell niche at progressively later times in development. This can be thought of as the cells being frozen in the stage of competence dictated by their embryonic history up to that point. Since vertebrates, especially amniotes like mammals, build up to enormous cell number, it would be expected that numerically most stem cells would be set aside later than earlier, and therefore most stem cells in foetal and post-natal stage tissues would be partially restricted. Thus in any tissue after the pre-embryo and early embryonic stage, few stem cells would have wide potency but most would be older stem cells having a restricted range of potencies, and this restriction would centre around the cell types in the tissue from which the stem cells were isolated.

This progressive restriction guides the development of specific cell types (the histotype) appropriate for the various tissues. However, in early embryonic development cells also acquire another form of information called positional information which specifies major body regions (Meinhardt, Dev Dyn 235:2907-2919, 2006). The same positional information is shared by cells of different histotype but of common spatial or regional origin. Positional information arises from exposure to positionally variable concentrations, durations and combinations of morphogens or growth factors (Ashe and Briscoe, Development 133:385-394, 2006). Positional information is retained by expression of combinations of positional patterning genes (Deschamps and van Nes, Development 132:2931-2942, 2005).

It is proposed that if stem cells are set aside at and after the stages of positional coding, they will have also acquired positional information. If more stem cells are set aside later than earlier, then most stem cells will have body region-specific positional information. Differentiated cells retain this positional information for the lifetime of the organism (Rinn et al, PLoS Genet 2(7):e119, 2006), and it is proposed that older stem cells likewise retain the positional coding already acquired.

Despite the potential of stem cell therapy to facilitate disease control in the nervous system and elsewhere (Webber and Minger, Curr Opin Investig Drugs 5(7):714-719, 2004), difficulties occur in relation to efficiency of appropriate cell differentiation and proliferation (Kieburtz and Olanow, supra 2007). Early embryonic stem cells require extensive and complex differentiation control to yield the desired cell histotypes (Biswas and Hutchins, supra 2007). Furthermore, differentiation into unwanted cells (“off target differentiation”) and even neoplasias are possible adverse outcomes (Hentze et al, Trends Biotechnol 25(1):24-32, 2007). The problems encountered can to some degree be addressed using partially restricted stem cells with the desired histotypic competence. Since such cells are of older origin, this potentially permits their clinical isolation from the patient, thereby avoiding immunological rejection problems. Older stem cells with partial restriction addresses the issue of histotypic appropriateness, and graft rejection but does not necessarily address the issue of positional or regional specificity.

A number of developmental disease conditions occur in the central and peripheral nervous systems which potentially could be treated by stem cell therapy (Webber and Minger, Curr Opin Investig Drugs; 5(7):714-719, 2004, Taupin, Indian J Med Res. 124(6):613-618, 2006). For example, the enteric nervous system (ENS) comprises the neurones and glial cells of the gastrointestinal tract, and is the largest part of the autonomic nervous system. Absence of the ganglia of the ENS (enteric aganglionosis, Hirschsprung's Disease or HS CR) is a relatively common and potentially fatal birth defect affecting 1/5000 live births, mostly males. In HSCR patients the ENS is normal through most of the intestine, and the disease usually affects only the distal colon. Lack of ENS in the distal colon produces intractable constipation and distension proximal to the aganglionic region (megacolon). Despite its abnormal appearance, the intestine itself is normal in most HSCR patients. HSCR is a well-defined clinical entity, resulting from mutations in many genes. HSCR is treated by removal of the affected bowel, although this leaves about half the patients with continence problems (Farlie et al, Birth Defects Res Part C Embryo Today 72:173-189, 2004; Young et al, In: Embryos, Genes and Birth Defects 2:263-300, 2006).

ENS cells arise as neural crest (NC) cells, a population including stem cells, that forms in association with the of the developing central nervous system all along the axis of the vertebrate body (teng and Labosky, Adv Exp Med Biol 589:206-212, 2006)). However, almost all ENS cells arise in a positionally restricted NC location, the brain stem sub-region of the cranial part of the NC, Early in gestation, these particular NC cells migrate to the gastrointestinal tract. Animal models show that HSCR is caused by defective migration of NC stem cells along the colon.

It is envisaged that NC stem cells may be used to replace the NC stem cell-derived cells missing in the colon in HSCR (Bums et al, Neurogastroenterol Motil. 16(1:3-7), 2004). NC stem cells can be isolated from a variety of locations (neural crest, intestine, peripheral nerve, skin, dental pulp, hair and whisker follicle) at a range of ages from embryonic to foetal to adult. These can be grown and expanded in vitro. However, NC stem cells are not all equal in ability to form various NC derivatives. These differences involve the age (Kruger et al, Neuron 35(4):657-669, 2002; Mosher et al, Dev Biol 303(1):1-15, 2007; Wong et al, J Cell Biol 175(6):l005-1015, 2006) and the position of the source tissue.

There is a need for improved protocols for stem cell therapy in relation to nervous system disorders in general and ENS disorders in particular, as well as in the repair, maintenance, regeneration or augmentation of other tissue types.

### SUMMARY

Early in embryonic development cells also acquire information guiding the type of cell (the histotype) they can become. They also acquire another form of information called positional information which specifies major body regions. The same positional information is shared by cells of different histotype but of common spatial or regional origin. It is proposed that if stem cells are set aside at and after the stages of positional coding, they will have also acquired positional information. If more stem cells are set aside later than earlier, then most stem cells will have body region-specific positional information. Differentiated cells retain this positional information for the lifetime of the organism, and it is proposed that stem cells likewise retain the positional coding already acquired.

It is postulated herein that stem cells are “frozen” in the stage of competence dictated by their embryonic history up to that point. Hence, in accordance with the present invention, successful stem cell therapy requires both histotypic (or cell type) competency and requisite positional competency. Non-histotypic positional information is established by positioned and temporal exposures to morphogens or growth factors (Ashe and Briscoe, supra, 2006) starting before gastrulation and continuing in neurulation and early organogenesis stages. Positional informations are preserved by the cell in patterns of gene expression (Deschamps and van Nes, surpa 2005). Hence, it is proposed herein that cells of the same histotype but different position are not identical. For example, cartilage cells from the primordium of the jaw are different from cartilage cells from the knee rudiment.

An improved stem cell therapeutic protocol includes choosing cells with the correct positional information. One method of selection of cells with the potential for the correct positional information includes the use of “fate maps” (Meinhardt, Dev Dyn 235:2907-2919, 2006). Fate maps have been developed based on the tagging a progenitor cells in the embryo with a dye or genetic marker in order to later identify its descendants (Lawson and Pedersen, Ciba Found Synmp 165:3-21, 1992). Cells with the identifying label are related to each other and therefore stems cells isolated from this population of cells have the correct positional information. Therefore, cells with the correct positional information as determined from fate maps can be targeted for isolation of stem cells for therapeutic protocols.

An improved stem cell therapeutic protocol is therefore provided for neuronal and non-neuronal cell repair, maintenance, regeneration and augmentation. The improvement comprises inter alia the use of a selected population or class of stem cells. The population or class of stem cells are positionally coded to permit differentiation to a target cell type appropriate for a specific region. Hence, positionally potent or spatially potent stem cells are contemplated for use in a stem cell therapeutic protocol. Such cells are referred to herein as “positiopotent” and “spatiopotent” stem cells meaning that the cells preferentially differentiate and proliferate into a target cell type. Hence, the cells are also referred to as proliferospatiohistocytotypiopotent stem cells.

In particular, a therapeutic stem cell protocol is provided employing histotypic competent and positional competent stem cells.

The use of such cells facilitates non-invasive tissue repair, maintenance, regeneration and/or augmentation.

Accordingly, a method provided is for conducting stem cell therapy in a subject, comprising isolating histotypic competent cells from the subject or compatible donor, which cells are positionally coded to permit differentiation to a target cell type to be generated, replaced, repaired or augmented, expanding the stem cells to generate an expanded population and then returning the expanded population of stem cells to the subject for a time and under conditions sufficient for the cells to differentiate to generate, replace, repair or augment the target cell types.

Another aspect contemplates a method of tissue or neuronal generation or replacement, repair or augmentation therapy in a subject, comprising isolating histotypic components stem cells from the subject or compatible donor which are spatiocompetent for the tissue or neurons to be replaced, repaired or augmented, expanding the stem cells in vitro and then administering the expanded stem cells to the subject under conditions which facilitate the therapy.

Still a further aspect relates to an improved method of stem cell therapy in a subject comprising collecting histotypic competent stem cells from the subject or compatible donor, expanding the stem cells in vitro and re-introducing the expanded stem cells to the subject, the improvement comprising selecting stem cells positionally coded to permit differentiation to form tissue or a neuronal cells which is the subject of therapy.

Yet another aspect provides a therapeutic protocol comprising identifying a condition in a subject requiring tissue or neuronal cell generation, replacement, repair or augmentation, isolating histotypic stem cells spatiocompetent to differentiate into the identified tissue or neuronal cells, expanding a population of isolated stem cells and administering the expanded cells to the subject.

In one embodiment the cells are part of the peripheral nervous system (PNS). This includes the enteric neural system (ENS). In another embodiment, the target cells are within the central nervous system (CNS). Even in yet another embodiment, the target cells are non-neural cells such as vascular or organ tissue cells.

The therapeutic protocol of the present invention is particularly useful in the non-surgical treatment of Hirschsprung's Disease (HSCR). Hence, a method for the treatment of HSCR is contemplated herein.

Any subject may be treated included humans and non-human animals. This includes birds, fish and amphibians.

Invasive and non-invasive stem cell collection protocols are contemplated herein such as collecting cells from hair follicles, skin and dental pulp.

### DETAILED DESCRIPTION

Throughout this specification, unless the context requires otherwise, the word “comprise”, or variations such as “comprises” or “comprising”, were understood to imply the inclusion of a stated element or integer or group of elements or integers but not the exclusion of any other element or integer or group of elements or integers.

All scientific citations, patents, patent applications and manufacturer's technical specifications referred to hereinafter are incorporated herein by reference in their entirety.

It is to be understood that unless otherwise indicated, the subject invention is not limited to specific formulation components, manufacturing methods, biological materials or reagents, dosage regimens and the like, as such may vary. It is also to be understood that the terminology used herein is for the purpose of describing particular embodiments only and is not intended to be limiting.

As used in the subject specification, the singular forms “a”, “an” and “the” include plural aspects unless the context clearly dictates otherwise. Thus, for example, reference to “a stem cell” includes a single cell, as well as two or more cells; reference to “an agent” or “a reagent” includes a single agent or reagent, as well as two or more agents or reagents;

reference to “the invention” or “an invention” includes single or multiple aspects of an invention; and so forth.

The terms “agent”, “reagent”, “compound”, “pharmacologically active agent”, “medicament”, “therapeutic”, “active” and “drug” are used interchangeably herein to refer to a chemical or biological entity which induces or exhibits a desired effect and all terms include a population of histotypic competent and positionally informed stem cells or one or more cytokines and/or growth factors which facilitate stem cell proliferation and differentiation.

Reference to an “agent”, “chemical agent”, “compound”, “pharmacologically active agent”, “medicament”, “therapeutic”, “active” and “drug” includes combinations of two or more active agents or two or more populations of cells. A “combination” also includes multi-part such as a two-part composition where the agents (including cells) are provided separately and given or dispensed separately or admixed together prior to dispensation.

The terms “effective amount” and “therapeutically effective amount” of an agent as used herein mean a sufficient amount of the agent to provide the desired therapeutic or physiological or effect or outcome. Such an effect or outcome includes the repair, maintenance, regeneration or augmentation of target tissue. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. for example from 10 to 1×10¹⁰ cells may be introduced or from 10 to 1×10¹⁰ cells/kg of patient body weight may be administered.

Hence, the present invention is predicated in part on the proposition that for appropriate stem cell therapy, stem cells need to be selected on the basis of cell type or histotypic competency and have the appropriate positional coding for the target tissue. Reference to “histotypic competency” means that the stem cells have undergone some differentiation towards the generic tissue or cell target. Reference to “positional coding” or “positional competency” means that the stem cell is anatomically competent to form a particular species of generic target cell or tissue.

For example, neural stem cells in the brain, consist of a diverse group of stem cells. These stem cells, depending on location, are capable of giving rise to only specific types of neurons cells; astrocytes, oligodendrocytes and neurons (Merkle et al, Science 317(5836):381-384, 2007; Klein et al, Development 132(20):4497-4508, 2005).

Hence, the present invention contemplates a method provided is for conducting stem cell therapy in a subject, comprising isolating histotypic competent cells from the subject or compatible donor, which cells are positionally coded to permit differentiation to a target cell type to be generated, replaced, repaired or augmented, expanding the stem cells to generate an expanded population and then returning the expanded population of stem cells to the subject for a time and under conditions sufficient for the cells to differentiate to generate, replace, repair or augment the target cell types.

Another aspect contemplates a method of tissue or neuronal generation or replacement, repair or augmentation therapy in a subject, comprising isolating histotypic components stem cells from the subject or compatible donor which are spatiocompetent for the tissue or neurons to be replaced, repaired or augmented, expanding the stem cells in vitro and then administering the expanded stem cells to the subject under conditions which facilitate the therapy.

A further aspect relates to an improved method of stem cell therapy in a subject comprising collecting histotypic competent stem cells from the subject or compatible donor, expanding the stem cells in vitro and re-introducing the expanded stem cells to the subject, the improvement comprising selecting stem cells positionally coded to permit differentiation to form tissue or a neuronal cells which is the subject of therapy.

Still another aspect provides a therapeutic protocol comprising identifying a condition in a subject requiring tissue or neuronal cell generation, replacement, repair or augmentation, isolating histotypic stem cells spatiocompetent to differentiate into the identified tissue or neuronal cells, expanding a population of isolated stem cells and administering the expanded cells to the subject.

Any type of stem cell is contemplated for use in accordance with the present invention. Examples of histotypic stem cells are provided in Table 1.

In addition, in the treatment of, for example, ENS-type disease conditions, neural crest cells (NC cells) are particularly contemplated. However, the NC cells or the cells listed in Table 1 are required to be positionally coded for the appropriate anatomical or nervous system location.

Hence, a population of histotypic competent and positionally competent stem cells is contemplated herein.

TABLE 1
Histotypic Stem Cells
Cell type

General Stem Cell Types
Embryonic stem cells
Somatic stem cells
Germ stem cells
Human embryonic stem cells
Human epidermal stem cells
Adipose derived stem cells
Brain
Adult neural stem cells
Human neurons
Human astrocytes
Epidermis
Human keratinocyte stem cells
Human keratinocyte transient amplifying cells
Human melanocyte stem cells
Human melanocytes
Skin
Human foreskin fibroblasts
Pancreas
Human duct cells
Human pancreatic islets
Human pancreatic β-cells
Kidney
Human adult renal stem cells
Human embryonic renal epithelial stem cells
Human kidney epithelial cells
Liver
Human hepatic oval cells
Human hepatocytes
Human bile duct epithelial cells
Human embryonic endodermal stem cells
Human adult hepatocyte stem cells (existence controversial)
Breast
Human mammary epithelial stem cells
Lung
Bone marrow-derived stem cells
Human lung fibroblasts
Human bronchial epithelial cells
Human alveolar type II pneumocytes
Muscle
Human skeletal muscle stem cells (satellite cells)
Heart
Human cardiomyocytes
Bone marrow mesenchymal stem cells
Simple Squamous Epithelial cells
Descending Aortic Endothelial cells
Aortic Arch Endothelial cells
Aortic Smooth Muscle cells
Eye
Limbal stem cells
Corneal epithelial cells
CD34+ hematopoietic stem cells
Mesenchymal stem cells
Osteoblasts (precursor is mesenchymal stem cell)
Peripheral blood mononuclear progenitor cells (hematopoietic stem cells)
Osteoclasts (precursor is above cell type)
Stromal cells
Spleen
Human splenic precursor stem cells
Human splenocytes
Immune cells
Human CD4+ T-cells
Human CD8+ T-cells
Human NK cells
Human monocytes
Human macrophages
Human dendritic cells
Human B-cells
Nose
Goblet cells (mucus secreting cells of the nose)
Pseudostriated ciliated columnar cells (located below olfactory region in
the nose)
Pseudostratified ciliated epithelium (cells that line the nasopharangeal
tubes)
Trachea
Stratified Epithelial cells (cells that line and structure the trachea)
Ciliated Columnar cells (cells that line and structure the trachea)
Goblet cells (cells that line and structure the trachea)
Basal cells (cells that line and structure the trachea)
Oesophagus
Cricopharyngeus muscle cells
Reproduction
Female primary follicles
Male spermatogonium

This improved stem cell therapeutic protocol for the selection of positionally competent cells includes a method of selection of cells. One method of selection of cells with the potential for the correct positional information includes the use of “fate maps”. Fate maps show the relationship of cells to each other based on their position in an embryo. Ie., Progenitor cells in the embryo are tagged with a dye or genetic marker in order to later identify its descendants. Cells with the identifying label are related to each other and therefore stems cells isolated from this population of cells have the correct positional information.

Histotypical competent and positionally competent cells may be selected by any number of means including “fate maps”, surface marker selection, FACS, DNA or methylation profiles, size sorting and may also be cultured in vitro in the presence of one or more cytokines or growth factors.

The term “subject” as used herein refers to an animal, and includes avian, amphibian and fish, preferably a mammal and more preferably a primate including a lower primate and even more preferably, a human who can benefit from the methods and assays of the present invention. A subject regardless of whether a human or non-human animal or embryo may be referred to as an individual, subject, animal, patient, host or recipient. The present invention therefore has both human and veterinary applications. For convenience, an “animal” specifically includes livestock species such as cattle, horses, sheep, pigs, camelids, goats and donkeys as well as avian, fish and amphibians. With respect to horses, these include horses used in the racing industry as well as those used recreationally or in the livestock industry.

Examples of laboratory test animals include mice, rats, rabbits, guinea pigs and hamsters. Rabbits and rodent animals, such as rats and mice, provide a convenient test system or animal model as do primates and lower primates.

The terms “disorder”, “abnormality” and “condition” may be used interchangeably to refer to an adverse health condition brought about by an alteration in the sequence of nucleotides or methylation patterns and/or a change in metabolic patterns in a subject. NC cell differentiation competence varies related to their anterior-posterior position of origin. It was long regarded that this pertained only to skeleton forming ability, which was restricted to the cranial NC. However, anterior-posterior positional restrictions also apply to the NC-derived nervous system (Table 2; first described by Newgreen et al, Cell Tissue Res 208:1-19, 1980). Cranial NC anterior to the vagal level has a similar ability to form ENS to the vagal level, even though these more anterior NC cells do not normally produce ENS. In contrast trunk NC, posterior to the vagal level, does not normally form an ENS and is not competent to do so.

TABLE 2
ENS fate and competence versus position of origin

| | | ENS | |
|---|---|---|---|
| NC Level | ENS Fate | Competence | Hox code |
| Midbrain | No | Yes | nil |
| Ant. hindbrain | No | Yes | Hox-1, 2 |
| Vagal (post. | Yes | Yes | Hox-3 |
| hindbrain) | | | |
| Cervical to lumbar | No | No (<2% vagal | Hox-6 to 8 |
| | | number) | |
| Sacral | Yes (low % of total) | No (<2% vagal | Hox-10 and |
| | | number) | above |

Like embryonic NC cells, foetal and post-natal NC stem cells of different positions of origin differ in their ability to generate various lineages (Table 3). For example, NC stem cells harvested from the sciatic nerve sheath cannot form ENS when back-grafted into younger embryos, although they can form both neurons and glial in cell culture. In contrast, NC stem cells obtained from the intestine can form ENS. However, positional information has longevity, and the inventors propose that ENS-forming competence in embryonic NC cells is spatially restricted even before these cells commence migration. Therefore, the inability of the sciatic-derived cells to form ENS is because their precursors in the trunk NC never acquired vagal/cranial positional information, not because the NC-derived cells were exposed to the environment of the sciatic nerve. Consistent with this, NC stem cells from similar tissues (hair follicles) but different positions (head and trunk) show differences in competence, the former having specific cranial NC-like competences that the latter lack.

TABLE 3
ENS Competence of Neural and NC Stem Cells of Various Sources

| | | | | Ability to | |
|---|---|---|---|---|---|
| | | | | form | |
| | | | | other | |
| | | | | cranial | |
| | Predicted | | Ability | specific | |
| Source of Stem | Positional | Age at | to form | NC | |
| Cells | Identity | derivation | ENS | derivs. | Reference |
| Embryonic NC | Cranial | Early | Yes | Yes | (Newgreen et al, |
| | | embryo | | | supra 1980) |
| Embryonic NC | Trunk | Early | No | No | (Newgreen et al, |
| | | embryo | | | supra 1980) |
| Intestinal NC | Cranial | Foetal and | Yes | Not | (Fu et al, J Cell |
| | | post-natal | | known | Biol 166: 673-684, |
| | | | | | 2004) |
| | | | | | (Bixby et al, |
| | | | | | Neuron 35: 643-656, |
| | | | | | 2002) |
| Sciatic nerve | Trunk | Foetal | No | Not | (Mosher et al, Dev |
| | | | | known | Biol 303: 1-15, |
| | | | | | 2007) |
| Whisker follicle | Cranial | Post-natal | Not | Yes | (Sieber-Blum et al, |
| | | | known | | Dev Dyn 231: 258-269, |
| | | | | | 2004) |
| Head hair | Cranial | Post-natal | Not | Yes | (Wong et al, supra, |
| follicle | | | known | | 2006) |
| Body hair | Trunk | Post-natal | Not | No | (Wong et al, supra |
| follicle | | | known | | 2006) |
| CNS | Cranial | Foetal | Yes | Not | (Micci et al, |
| | | | | known | Gastroenterology |
| | | | | | 121: 757-766, 2001) |

Although the present invention is applicable to the repair, maintenance, regeneration or augmentation of any tissue or cell type, one particular condition is the treatment of HSCR in infants. This treatment involves the use of histotypic competent and positional competent NC cells.

The present invention is now described in relation to the following non-limiting Examples. In these Examples, materials and methods as outlined below may be employed.

(i) Harvesting ENS NC stem cells. As a positive control cell, NC stem cells obtained from neurospheres derived from mouse embryonic intestinal tissue are used. These NC stem cells are of known ENS forming ability.

(ii) Harvesting NC cells. As further control experimental cells, the ENS-forming ability of bone fide primary NC cells from mouse embryos is determined. These are harvested by Dispase assisted microdissection from C57 black mouse embryos at embryonic day (E) 8.5 to 10.5, as detailed in the methods review by Newgreen and Murphy, Methods Mol Biol 137:201-211, 2000. The anterior to posterior level obtained varies from midbrain, vagal, thoracic to sacral depending on the embryonic age. It is proposed that the different levels will show drastically different ENS-forming abilities depending on position of origin, as is observed for similar cells from avian embryos.

(iii) Harvesting NC stem cells from hair follicles. The so-called bulge region of hair follicles is a source of NC stem cells. The NC stem cells obtained from facial whisker follicles gives rise to the entire repertoire of cranial NC cells, including neurones, Schwann cells, melanocytes, and also the cranial-specific smooth muscle cells and chondrocytes (Sieber-Blum et al, Birth Defects Res C Embryo Today 72:162-172, 2004).

Whisker and hair follicles are dissected from various regions of the skin of 2 month (approx.) C57 mice. Since epidermal NC stem cells in different regions are derived from local NC cells, they must share positional information appropriate to their site of NC origin. Follicles from cranial and facial, upper neck, and trunk (between the limbs) sites will be compared to assay a spread of positional determinants.

The dermis and fat are removed with tungsten needles and with buffer rinses, exposing the ring sinus below the skin and the cavernous sinus near the base of the follicle. This region is excised and the capsule is cut lengthwise. This gives the bulge area within a connective tissue capsule. The bulge region is squeezed out of the capsule: it forms a structure about 100×300 micrometres.

The bulge is placed into a collagen-coated culture plate, where it rapidly adheres.

(iv) Tissue culture. Culture medium is 75% alpha-modified MEM medium, 5% day 11 chick embryo extract, and 10% of foetal calf serum, and 1 microg/ml gentamycin (see Sieber-Blum et al., 2004). Culture medium is half-changed every second day. After 4 days, the explant is removed leaving a halo of 100-150 migrating cells.

(v) Confirmation of NC status. It is reported that these follicular bulge-derived halo cells are all NC derived cells (Sieber-Blum et al, supra 2004). This is confirmed by antibody staining with known NC reporters: nestin, Sox10 and p75 antibodies for undifferentiated NC cells including stem cells, and for possible NC differentiation products: Tuj1, HuC/D, nNOS (neurons); GFAP, BFABP, S100 (glial cells); Mel (melanocytes); desmin, SMA (smooth muscle) and collagen type II (cartilage). At this stage, these differentiated cells are unlikely to be numerous. Characterization of NC and ENS cells by immunolabelling is a standard technique.

(vi) Preparation of NC cell carriers. The same procedure as above is carried out and then the halo cells are lifted (0.005% trypsin) and replated onto 3 microlitre collagen gel plugs moulded into non-TC Terasaki wells, at about 100 cells per 10 microlitre well. After growth to several thousand cells (over about 4-6 days; cell cycle is initially around 6 hours) the collagen gel plug plus cells is removed by ringing with a tungsten needle. This provides, on a convenient carrier, a group of NC stem cells whose positional origin is defined by the site from which the follicle was obtained.

(vii) Test of ENS-forming competence. The collagen plug with follicle-derived NC stern cells is abutted to the cut end of an aneural hindgut of E11.5 donor mouse embryo in “catenary” culture (Hearn et al, Dev. Dyn. 214:239-247, 1999). Catenary cultures are used for developmental ENS and general intestinal studies. This system facilitates colonization of the intestine by competent NC-derived cells. It also allows differentiation into neurons and glia, assembly into ganglia and development of neurite connections. In innervated catenary guts, there is even evidence of emergence of peristalsis-like intestinal contractions.

(viii) Predicted results. The predicted outcome is that the neurosphere derived NC stem cells and all cranial level embryonic NC cells furnish aneural hindgut with an ENS over a four day culture period. That is, the intestinal explants have NC derived cells throughout, some of these will express markers for neuronal and glial differentiation, and assemble into ganglia which will extend nerve fibres. But the trunk embryonic NC cells will be incapable of generating an ENS, although some cells will be found in the intestinal wall. It is proposed that the follicle-derived NC stem cells will be able to furnish an ENS, but this will be restricted to cells derived from follicles of the whiskers and head hair, that is, those of cranial origin and cranial positional identity.

### EXAMPLE 1

### Generation of ENS

Different sources of stem cells to form an ENS in the aganglionic region of newborn mice in vivo. Details of the sources of stem cells, recipient aganglionic gut, the introduction of stem cells into the recipient gut and the analysis are described separately below. All mice (from which stem cells are obtained and recipient) are on a C57B1/6 background.

### EXAMPLE 2

### Sources of Neural and NC Stem Cells

All neural and NC stem cells express green fluorescent protein (GFP) either in the nucleus of all cells or in the cytoplasm (driven by the Ret promoter, see Young et al, Dev Biol 270:455-473, 2004. Expression of GFP permits rapid initial screening of ENS formation before selection for more detailed analysis of ENS cell types.

(i) Embryonic NC cells from different anteroposterior levels of the neural axis. As control experimental cells the ENS-forming ability of bone fide primary NC cells from mouse embryos is tested. NC cells are harvested from cultured neural tube explants from E8-E10.5 nuclear GFP mouse embryos as described previously (Newgreen and Murphy, supra 2000). The anterior to posterior level obtained varies from midbrain to sacral depending on the embryonic age. It is proposed that different anteroposterior levels will show drastically different ENS-forming abilities as is observed for similar cells from avian embryos.

(ii) ENS NC stem cells. One source of ENS-competent NC stem cells is the bowel. Three types of ENS NC stem cells are examined: 1. NC stem cells obtained from neurospheres derived from mouse embryonic intestinal tissue. NC stem cells are of known ENS forming ability in embryonic gut co-culture assays (Pu et al, supra 2004). 2. NC stem cells obtained from the bowel, at various ages, using expression of high levels of α4 integrin and p75 to obtain highly enriched enteric NC stem cells. 3. A mixed population, including NC stem cells and more restricted lineages, defined by Ret expression. Ret is a receptor tyrosine kinase that is critical for ENS development. NC-derived cells are the only cells in the gut to express Ret, and all NC-derived cells in the intestine express Ret (Young et al, Dev Dyn 216:137-152, 1999). To obtain high a4 integrin+/p75+ cells the gut from E12.5 nuclear GFP mice will be dissected, dissociated and the high α4 integrin+/p75+ cells will be isolated using antibodies against a4 integrin and p75 and fluorescence-activated cell sorting (FACS) [Bixby et al, supra 2002]. To isolate Ret+ cells, the gut from E12.5 Ret-GFP mice are dissected, dissociated and GFP-expressing cells isolated using FACS. The ability of enteric neurospheres, cells expressing high α4 integrin/high p75, and Ret⁺ cells to form an ENS in aganglionic segments of post-natal mouse gut in vivo are compared.

The status of the isolated cells is confirmed by antibody staining with known NC reporters: nestin, Sox10, Ret and p75 antibodies for undifferentiated NC cells including stem cells, and for possible NC differentiation products: Tuj1, HuC/D, nNOS (neurons); GFAP, B-FABP, S100b (glial cells); Mel (melanocytes); desmin, SMA (smooth muscle) and collagen type II (cartilage). Characterization of NC ENS cells by immunolabelling is a standard technique in our laboratories (Young et al, Cell Tissue Res 320:1-9, 2005).

(iii) NC stem cells from hair follicles. NC stem cells can be readily isolated from epidermal hair follicles, and offer the unique clinical advantage of non-invasive collection from a post-natal donor. The so-called bulge region of hair follicles is a source of NC stem cells. The NC stem cells obtained from facial whisker follicles and head hair can give rise to a very wide (possibly entire) repertoire of cranial NC cells, including neurons, Schwann cells, melanocytes, and also the cranial-specific smooth muscle cells and chondrocytes (Sieber-Blum et al, supra 2004; Wong et al, supra 2006). Follicular NC stem cells from the trunk have a more restricted repertoire (Wong et al, supra 2006). Neither source of NC stem cell has been tested specifically for ENS competence.

Since epidermal hair follicle NC stem cells in different regions are derived from local NC cells, they must share positional information appropriate to their site of NC origin. Follicles from cranio-facial, and trunk (between the limbs) skin of two month old nuclear GFP mice will be compared to assay a spread of positional determinants.

The dermis and fat is removed with tungsten needles, exposing the ring sinus below the skin and the cavernous sinus near the base of the follicle. This region will be excised and the capsule cut lengthwise. This gives the bulge area within a connective tissue capsule. The bulge region will be squeezed out of the capsule: it forms a structure about 100×300 micrometres. NC stem cells can then be obtained by two methods: 1. Tissue culture: The bulge will be placed onto a collagen-coated culture plate with culture medium includes 75% alpha-modified MEM medium, 5% day 11 chick embryo extract and 10% foetal calf serum (see Sieber-Blum et al, supra 2004). After four days, the explant will be removed leaving a halo of 100-150 migrating cells. These follicular bulge-derived halo cells are all NC derived cells (Sieber-Blum et al, supra 2004). The NC status will be confirmed by antibody staining with known NC reporters as above. This population readily expands in vitro, with an initial cell cycle time of 6 hours. 2. Fluorescence-activated cell sorting (FACS). This will be performed on trypsin/EDTA dissociated hair follicle cells using antibodies to α4 integrin and p75 as described above for the isolation of enteric NC stem cells.

### EXAMPLE 3

### Recipient Gut

Mice lacking endothelin-3 (Et3) lack enteric neurons in the distal 20 mm of the bowel. Some humans with HSCR have mutations in ET3, and thus Et3−/− mice are an accepted model of HSCR. P0-P3 mice with be anaesthetized with halothane. An abdominal incision is made, and the distal colon exposed. Stem cells are injected into the distal colon. At P0-P3, Et3−/− mice are not phenotypically distinguishable from Et3+/− and Et3+/+ mice, DNA is therefore extracted from samples of tails tips. Hence, stem cells are injected into the distal colon of both aganglionic (Et3−/−) and normo-ganglionic (Et3+/+ and Et3+/−) mice. The incision is sutured, and the mice killed 14 days later. Genotyping using PCR of Et3+/+, Et3+/− and Et3−/− mice is performed using standard procedures.

### EXAMPLE 4

### Introduction of Stem Cells Into Recipient Gut

GFP+ stem cells in tissue culture medium is introduced into the gut of P0-P3 Et3+/+, Et3+/− and Et3−/− mice using a glass micropipette attached to a 10 μl Hamilton syringe. The tip of the pipette is slid through the serosa and advanced into the external muscle, then withdrawn slightly to create some space for the injection. Each injection is approximately 0.2 μl and contain 100-500 GFP+ cells. Initially a single injection or several widely spaced injections is made to judge the radial spread of injected cells. Then, to achieve greater coverage, multiple injections will be made around the circumference of the colon and along the terminal 20 mm of the colon at, for example, 3-5 mm intervals. Control injections of 0.2 μl of tissue culture medium only is also made.

### EXAMPLE 5

### Analysis

Analysis of ENS structure: 14 days after the introduction of stem cells, control and stem cell injected mice are killed. The colon are removed, opened along the mesenteric border, pinned flat onto balsa wood and fixed. Wholemount preparations of external muscle of the distal 50 mm of colon will be prepared and screened for GFP+ cells. This includes the aganglionic region plus regions containing an ENS in control Et3−/− mice. The tissue is processed for immunohistochemistry using antibodies to GFP to reveal the distribution and number of GFP+ cells in both aganglionic and control bowel. The distance that GFP+ cells have migrated away from the injection site in aganglionic and control bowel is examined as is the distribution of GFP+ cells in the aganglionic regions. Antibodies are used to Hu (a pan-neuronal marker) and to S100b (a glial marker) to determine the proportion of GFP+ cells that express neuronal or glial markers. If neurons are present, whether the major sub-types of enteric neurons occur is determined.

Analysis of ENS function: functional studies are conducted in vitro using the colon of Et3−/− mice in which an ENS is generated from stem cells to determine whether spontaneous propagating motility patterns are present and have the same characteristics as those in wild-type mice.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### Bibliography

Ashe and Briscoe, Development 133:385-394, 2006
Biswas and Hutchins, Stem Cells Div 16(2):213-222, 2007
Bixby et al, Neuron 35:643-656, 2002
Burns et al, Neurogastroenterol Motil. 16(1:3-7), 2004
Crane and Trainor, Annu Rev Cell Dev Biol 22:267-296, 2006
Deschamps and van Nes, Development 132:2931-2942, 2005
Farlie et al, Birth Defects Res Part C Embryo Today 72:173-189, 2004
Fu et al, J Cell Biol 166:673-684, 2004
Hearn et al, Dev. Dyn. 214:239-247, 1999
Hentze et al, Trends Biotechnol 25(1):24-32, 2007
Kieburtz and Olanow, Mt Sinai J Med 74(1):7-14, 2007
Klein et al, Development 132(20):4497-4508, 2005
Kruger et al, Neuron 35(4):657-669, 2002
Labosky, Adv Exp Med Biol 589:206-212, 2006
Larru, Trends Biotechnol 19(12):487, 2001
Lawson and Pedersen, Ciba Found Synmp 165:3-21, 1992
Meinhardt, Dev Dyn 235:2907-2919, 2006
Merkle et al, Science 317(5836):381-384, 2007
Micci et al, Gastroenterology 121:757-766, 2001
Mosher et al, Dev Biol 303:1-15, 2007
Newgreen et al, Cell Tissue Res 208:1-19, 1980
Newgreen and Murphy, Methods Mol Biol 137:201-211, 2000
Rinn et al, PLoS Genet 2(7):e119, 2006
Sieber-Blum et al, Dev Dyn 231:258-269, 2004
Sieber-Blum et al, Birth Defects Res C Embryo Today 72:162-172, 2004
Taupin, Indian J Med Res. 124(6):613-618, 2006
Webber and Minger, Curr Opin Investig Drugs; 5(7):714-719, 2004
Wong et al, J. Cell Biol. 175:1005-1015, 2006
Young et al, Dev Dyn 216:137-152, 1999
Young et al, Dev Biol 270:455-473, 2004
Young et al, Cell Tissue Res 320:1-9, 2005
Young et al, In: Embryos, Genes and Birth Defects 2:263-300, 2006

1. A method of conducting stem cell therapy in a subject, said method comprising isolating histotypic competent stem cells from said subject or compatible donor, said cells comprising stem cells which are positionally coded to permit differentiation to target cell types to be generated or undergoing replacement, repair or augmentation, expanding said stem cells to generate an expanded population and then returning the expanded population of stem cells to the subject for a time and under conditions sufficient for the cells to differentiate to generate, replace, repair or augment the target cell types.

2. The method of claim 1 wherein the target cell types comprise organ cells or vascular cells.

3. The method of claim 1 wherein the target cell types comprise neurons in the enteric neural system (ENS).

4. The method of claim 1 wherein the target cell types comprise neurons in the central nervous system (CNS).

5. The method of claim 1 wherein the target cell types comprise neurons in the peripheral neurons system (PNS).

6. The method of claim 1 or 3 wherein the stem cells are neural crest (NC) cells.

7. The method of claim 6 wherein the NC cells are positionally coded to permit differentiation into distal intestinal neurons.

8. The method of claim 7 wherein the NC cells are from cranial hair follicles.

9. The method of claim 7 for treating Hirschsprung's disease.

10. The method of claim 1 wherein the subject is a human.

11. A method of tissue or neuronal generation or replacement, repair or augmentation therapy in a subject, said method comprising isolating histotypic competent stem cells from said subject or compatible donor which are spatiocompetent for the tissue or neurons to be replaced, repaired or augmented, expanding the stem cells in vitro and then administering the expanded stem cells to the subject under conditions which facilitate the therapy.

12. The method of claim 11 wherein the tissue is organ or vascular tissue.

13. The method of claim 11 wherein the neurons are in the ENS.

14. The method of claim 11 wherein the neurons are in the CNS.

15. The method of claim 11 wherein the neurons are in the PNS.

16. The method of claim 11 wherein the stem cells are NC cells.

17. The method of claim 16 wherein the NC cells are positionally coded to permit differentiation into distal intestinal neurons.

18. The method of claim 17 wherein the NC cells are from cranial hair follicles.

19. The method of claim 17 in the treatment of Hirschsprung's disease.

20. The method of any one of claims 11 to 19 claim 11 wherein the subject is a human.

21. A method of stem cell therapy in a subject comprising collecting stem cells from said subject or compatible donor, expanding said stem cells in vitro and re-introducing the expanded stem cells to said subject, the improvement comprising selecting histotypic competent stem cells which are positionally coded to permit differentiation to form tissue or a neuronal cells which is the subject of therapy.

22. The method of claim 21 wherein the tissue is organ or vascular tissue.

23. The method of claim 21 wherein the neuronal cells are in the ENS.

24. The method of claim 21 wherein the neuronal cells are in the CNS.

25. The method of claim 21 wherein the neuronal cells are in the PNS.

26. The method of claim 21 wherein the stem cells comprise NC cells.

27. The method of claim 26 wherein the NC cells are collected from cranial hair follicles.)

28. The method of claim 26 in the treatment of Hirschsprung's disease.

29. The method of claim 21 wherein the subject is a human.

30.-38. (canceled)

39. A therapeutic protocol comprising identifying a condition in a subject requiring tissue or neuronal cell generation, replacement, repair or augmentation, isolating histotypic competent stem cells spatiocompetent to differentiate into the identified tissue or neuronal cells, expanding a population of isolated stem cells and administering said expanded cells to the subject.

40. The therapeutic protocol of claim 39 wherein the tissue is organ or vascular tissue.

41. The therapeutic protocol of claim 39 wherein the neuronal cells are from the ENS.

42. The therapeutic protocol of claim 39 wherein the neuronal cells are from the CNS.

43. The therapeutic protocol of claim 39 wherein the neuronal cells are from the PNS.

44. The therapeutic protocol of claim 39 wherein stem cells are NC cells.

45. The therapeutic protocol of claim 44 wherein the NC cells are derived from cranial hair follicles.

46. The therapeutic protocol of claim 44 in the treatment of Hirschsprung's disease.

47. The therapeutic protocol of claim 39 wherein the subject is a human.

48. A method for the non-surgical treatment of Hirschsprung's disease is a human infant, said method comprising isolating histotypic competent NC cells from cranial hair follicles from said infant or a compatible donor, expanding to NC cells in in vitro cultures and introducing the expanded NC cells to one or more sites in the intestine to permit generation of neuronal cells in the distal intestine.
